(19) United States
(12) Patent Application Publication
(10) Pub. No.: US 2025/0374968 A1
(43) Pub. Date: Dec. 11, 2025

(54) INHALER

(71) Applicant: KT&G CORPORATION, Daejeon (KR)

(72) Inventors: Min Kyu KIM, Seoul (KR); Paul Joon SUNWOO, Seoul (KR); Won Kyeong LEE, Gyeonggi-do (KR)

(73) Assignee: KT&G CORPORATION, Daejeon (KR)

(21) Appl. No.: 18/876,554

(22) PCT Filed: Aug. 24, 2023
(86) PCT No.: PCT/KR2023/012542

§ 371 Date: Dec. 18, 2024

(30) Foreign Application Priority Data
Sep. 6, 2022 (KR) 10-2022-0112636
Nov. 17, 2022 (KR) 10-2022-0154438

(51) Int. Cl. A24F 40/50; A24F 40/20; A24F 40/42; A24F 40/485

(52) U.S. Cl. CPC A24F 40/50; A24F 40/20; A24F 40/42; A24F 40/485

(57) ABSTRACT

An inhaler according to an embodiment includes a stick including a chamber configured to accommodate a capsule containing powder and a piercing hole that opens toward the chamber, a holder including an insertion groove into which the stick is inserted in a first direction, a piercing member provided in the insertion groove and configured to crush the capsule through the piercing hole when the stick is inserted into the insertion groove, and a vibrating member configured to provide vibration to the piercing member.

### TECHNICAL FIELD

Various embodiments of the present disclosure relate to an inhaler.

### BACKGROUND ART

Recently, demands for alternative articles to overcome disadvantages of general cigarettes have increased. For example, an inhaler is a device for inhaling a liquid or gas containing a composition such as a drug by a user through the oral cavity or nasal cavity of the user.

Such a device may have a chamber accommodating an inhalable composition, and the inhalable composition may pass from the chamber through a channel and finally to the oral or nasal cavity of a user to be inhaled by the user.

The above description has been possessed or acquired by the inventor(s) in the course of conceiving the present disclosure and is not necessarily an art publicly known before the present application is filed.

### DISCLOSURE OF THE INVENTION

### Technical Goals

In an inhaler using a composition in a powder state, a non-electronic inhaler according to the related art has to inhale the powder depending on the breathing of a user. In this case, the amount of powder discharged from the inhaler may vary according to the lung capacity of the user, and a user whose lung capacity does not meet a certain level has a problem in that the use of the inhaler is restricted.

To solve this problem, there is a demand for an inhaler capable of smoothly inhaling even by a user with weak lung capacity, and furthermore, individually controlling a discharged state of powder to a general user.

### Technical Solutions

According to an embodiment, an inhaler includes a stick including a chamber configured to accommodate a capsule containing powder and a piercing hole that opens toward the chamber, a holder including an insertion groove into which the stick is inserted in a first direction, a piercing member provided in the insertion groove and configured to crush the capsule through the piercing hole when the stick is inserted into the insertion groove, and a vibrating member configured to provide vibration to the piercing member.

In an embodiment, the vibrating member may be configured to vibrate the piercing member in a direction substantially parallel to the first direction.

In an embodiment, the vibrating member may be configured to vibrate the piercing member in a direction substantially perpendicular to the first direction.

In an embodiment, the vibrating member may be configured to vibrate the piercing member in a direction substantially parallel to the first direction and in a direction substantially perpendicular to the first direction.

In an embodiment, the inhaler may further include a puff sensor configured to sense airflow inside the stick and a processor configured to receive a sensing result from the puff sensor and control vibration of the vibrating member based on the sensing result.

In an embodiment, the inhaler may further include a sub-vibrating member configured to provide vibration to the chamber.

In an embodiment, the inhaler may further include an elastic member provided in the insertion groove and configured to be pressed by the stick when the stick is inserted into the insertion groove. The sub-vibrating member may be configured to provide vibration to the chamber of the stick through the elastic member.

In an embodiment, the sub-vibrating member may be configured to vibrate the chamber in a direction substantially parallel to the first direction.

In an embodiment, the sub-vibrating member may be configured to vibrate the chamber in a direction substantially perpendicular to the first direction.

In an embodiment, the sub-vibrating member may be configured to vibrate the chamber in a direction substantially parallel to the first direction and in a direction substantially perpendicular to the first direction.

In an embodiment, the inhaler may further include a puff sensor configured to sense airflow inside the stick and a processor configured to receive a sensing result from the puff sensor and control vibration of the vibrating member and the sub-vibrating member based on the sensing result.

In an embodiment, the stick may include a mouthpiece provided on an end portion opposite to the chamber, an airflow channel configured to provide fluid communication between the chamber and the mouthpiece, and a mesh arranged between the airflow channel and the chamber.

In an embodiment, the stick may further include a sealing member configured to seal the piercing hole and be crushed by the piercing member when the stick is inserted into the insertion groove.

In an embodiment, the stick may include a door configured to selectively open and close the piercing hole.

In an embodiment, the inhaler may further include an insertion detection sensor configured to detect whether the stick is inserted into the insertion groove, a door hinge configured to move the door, and a processor configured to receive a detection result from the insertion detection sensor and open and close the door by controlling the door hinge based on the detection result.

### Effects

According to an embodiment, an inhaler including at least one of a vibrating member and/or a sub-vibrating member may control the amount and/or rate of powder discharged from a capsule and assist a user in smoothly inhaling powder.

The effects of the inhaler are not limited to the above-mentioned effects, and other unmentioned effects can be clearly understood from the following description by one of ordinary skill in the art to which the present disclosure pertains.

### BRIEF DESCRIPTION OF DRAWINGS

FIG. 1 is a block diagram illustrating an inhaler according to an embodiment.

FIG. 2 is a diagram schematically illustrating an inhaler according to an embodiment.

FIG. 3a is a diagram schematically illustrating the inside of an inhaler according to an embodiment.

FIG. 3b is a diagram schematically illustrating the inside of an inhaler according to an embodiment.

FIG. 4a is a diagram schematically illustrating the inside of an inhaler according to an embodiment.

FIG. 4b is a diagram schematically illustrating the inside of an inhaler according to an embodiment.

FIG. 5a is a cross-sectional view of a stick according to an embodiment.

FIG. 5b is a cross-sectional view of a stick according to another embodiment.

### BEST MODE FOR CARRYING OUT THE INVENTION

The terms used in various embodiments are selected from among common terms that are currently widely used, in consideration of their function in the disclosure. However, the terms may become different according to an intention of one of ordinary skill in the art, a precedent, or the advent of new technology. Also, in particular cases, the terms are discretionally selected by the applicant of the disclosure, and the meaning of those terms will be described in detail in the corresponding part of the detailed description. Therefore, the terms used in the disclosure are not merely designations of the terms, but the terms are defined based on the meaning of the terms and content throughout the disclosure.

It will be understood that when a certain part “includes” a certain component, the part does not exclude another component but may further include another component, unless the context clearly dictates otherwise. Also, terms such as “unit,” “module,” etc., as used in the specification may refer to a part for processing at least one function or operation and which may be implemented as hardware, software, or a combination of hardware and software.

As used herein, an expression such as “at least one of” that precedes listed components modifies not each of the listed components but all the listed components. For example, the expressions “at least one of a, b, or c” and “at least one of a, b, and c” should be construed as including a, b, c, a and b, a and c, b and c, or a, b, and c.

In various embodiments, the term “puff” refers to inhalation by a user, and inhalation refers to a situation in which a user draws in an aerosol into his or her oral cavity, nasal cavity, or lungs through the mouth or nose.

In an embodiment, an inhaler may include a main body (or a holder) configured to support a cartridge (or a stick) configured to accommodate a capsule containing a composition. The cartridge may be detachably coupled to the main body. However, embodiments are not limited thereto. The cartridge may be integrally formed or assembled with the main body, and may be secured to the main body so as not to be detached by a user. The cartridge may be mounted on the main body while the capsule is accommodated therein. However, embodiments are not limited thereto. The powder or capsules containing the powder may be injected into the cartridge while the cartridge is coupled to the main body.

Hereinafter, embodiments of the disclosure will be described in detail with reference to the accompanying drawings such that one of ordinary skill in the art may easily practice the disclosure. The disclosure may be practiced in forms that are implementable in the inhaler according to various embodiments described above or may be embodied and practiced in many different forms and is not limited to the embodiments described herein.

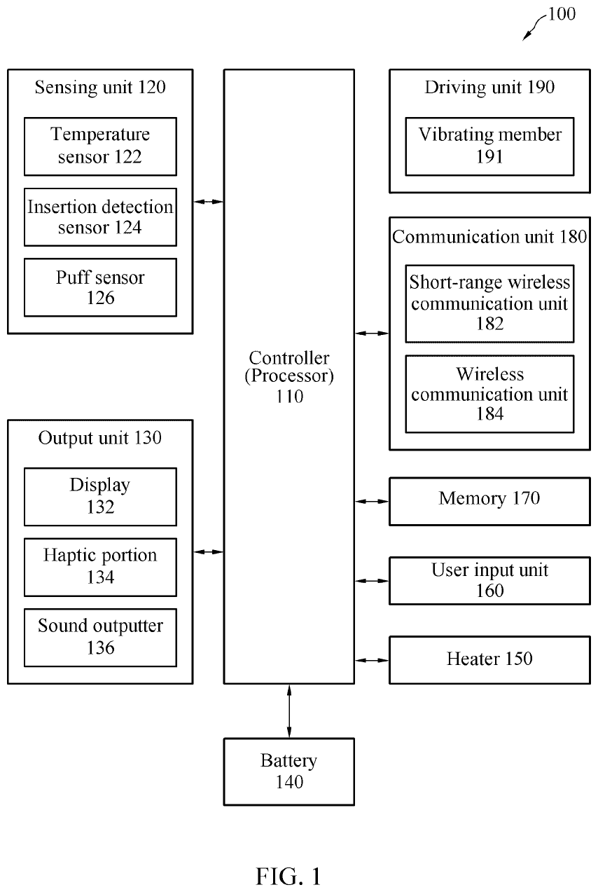

FIG. 1 is a block diagram illustrating an inhaler 100 according to an embodiment.

Referring to FIG. 1, the inhaler 100 according to an embodiment may include at least a portion of a controller 110, a sensing unit 120, an output unit 130, a battery 140, a heater 150, a user input unit 160, a memory 170, a communication unit 180, and a driving unit 190.

However, the internal structure of the inhaler 100 is not limited to that as shown in FIG. 1. It is to be understood by one of ordinary skill in the art to which the present disclosure pertains that some of the components shown in FIG. 1 may be omitted or new components may be added thereto according to the design of the inhaler 100

In an embodiment, the sensing unit 120 may sense a state of the inhaler 100 or a state of surroundings the inhaler 100 and transmit sensed information to the controller 110 (or a processor). The controller 110 may control driving of other components of the inhaler 100 based on the sensed information.

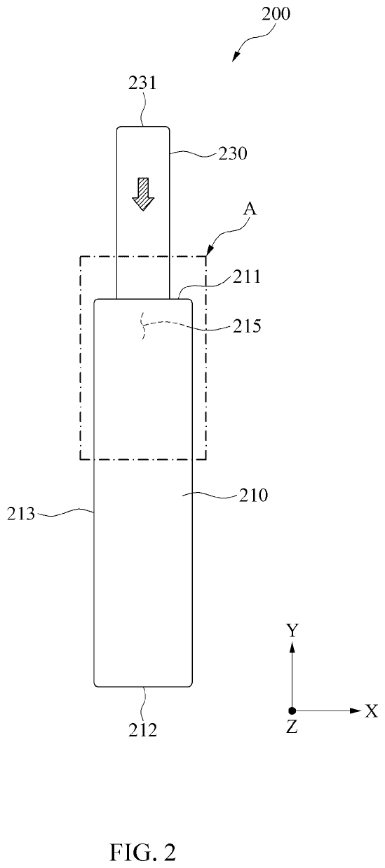

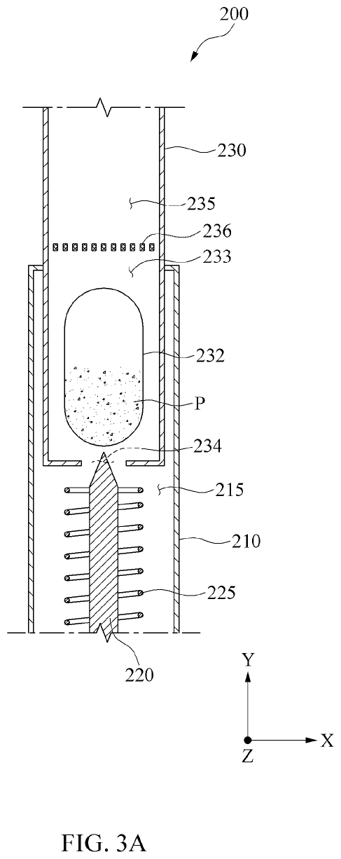

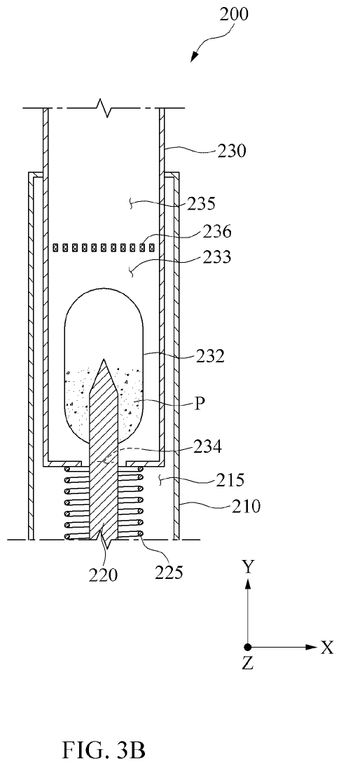

For example, the controller 110 may control an operation of the heater 150 based on a sensing result of the sensing unit 120, control the driving unit 190 by determining whether a stick (e.g., a stick 230 of FIG. 2), a capsule (e.g., a capsule 232 of FIG. 3), a cartridge, or a cigarette is inserted into an insertion groove, or perform various functions such as displaying a notification by the output unit 130.

In an embodiment, the sensing unit 120 may include at least one of a temperature sensor 122, an insertion detection sensor 124, or a puff sensor 126. However, embodiments are not limited thereto.

In an embodiment, the temperature sensor 122 may sense a temperature at which the heater 150 is heated. The inhaler 100 may include a separate temperature sensor for sensing the temperature of the heater 150, or the heater 150 itself may perform a function as a temperature sensor. Alternatively, the temperature sensor 122 may be arranged around the battery 140 to monitor the temperature of the battery 140.

In an embodiment, the insertion detection sensor 124 may detect insertion and/or removal of a stick (e.g., the stick 230 of FIG. 2) or a capsule (e.g., the capsule 232 of FIGS. 3a and 3b). The insertion detection sensor 124 may include, for example, at least one of a film sensor, a pressure sensor, a light sensor, a resistive sensor, a capacitive sensor, an inductive sensor, or an infrared sensor and may sense a signal change by the insertion and/or removal of the stick or capsule.

In an embodiment, the puff sensor 126 may sense a puff from a user based on various physical changes in an airflow path or airflow channel. For example, the puff sensor 126 may sense the puff from the user based on one of a temperature change, a flow change, a voltage change, and a pressure change.

In an embodiment, the sensing unit 120 may further include at least one of a temperature/humidity sensor, an atmospheric pressure sensor, a magnetic sensor, an acceleration sensor, a gyroscope sensor, a position sensor (e.g., a global positioning system (GPS)), a proximity sensor, or a red, green, blue (RGB) sensor (e.g., an illuminance sensor), in addition to the sensors described above. A function of each sensor may be intuitively inferable from its name by one of ordinary skill in the art, and thus, a detailed description thereof is omitted herein.

In an embodiment, the output unit 130 may output information about a state of the inhaler 100 and provide the information to the user. The output unit 130 may include at least one of a display 132, a haptic portion 134, or a sound outputter 136. However, embodiments are not limited thereto. When the display 132 and a touchpad are provided in a layered structure to form a touchscreen, the display 132 may be used as an input device in addition to an output device.

In an embodiment, the display 132 may visually provide information about the inhaler 100 to the user. For example, the information about the inhaler 100 may include at least a portion of various pieces of information such as a charging/discharging state of the battery 140 of the inhaler 100, a preheating state of the heater 150, an insertion/removal state of a stick or capsule, or a state in which the use of the inhaler 100 is restricted (e.g., detection of an abnormal item), or a vibration state of the driving unit 190, and the display 132 may externally output this information. The display 132 may be, for example, a liquid-crystal display (LCD) panel, an organic light-emitting display (OLED) panel, and the like. The display 132 may also be in the form of a light-emitting diode (LED) device.

In an embodiment, the haptic portion 134 may provide information about the inhaler 100 to the user in a haptic way by converting an electrical signal into a mechanical stimulus or an electrical stimulus. The haptic portion 134 may include, for example, a motor, a piezoelectric element, or an electrical stimulation device.

In an embodiment, the sound outputter 136 may provide the information about the inhaler 100 to the user in an auditory way. For example, the sound outputter 136 may convert an electrical signal into a sound signal and externally output the sound signal.

In an embodiment, the battery 140 may supply power to be used to operate the inhaler 100. The battery 140 may supply power to heat the heater 150.

In an embodiment, the battery 140 may supply power required for operations of the other components (e.g., the sensing unit 120, the output unit 130, the user input unit 160, the memory 170, the communication unit 180, or the driving unit 190) provided in the inhaler 100. The battery 140 may be a rechargeable battery or a disposable battery. The battery 140 may be, for example, a lithium polymer (LiPoly) battery. However, embodiments are not limited thereto.

In an embodiment, the heater 150 may receive power from the battery 140 to heat an aerosol generating material. Although not shown in FIG. 1, the inhaler 100 may further include a power conversion circuit (e.g., a direct current (DC)-to-DC (DC/DC) converter) that converts power of the battery 140 and supplies the power to the heater 150. In addition, when the inhaler 100 generates an aerosol in an induction heating manner, the inhaler 100 may further include a DC-to-alternating current (AC) (DC/AC) converter that converts DC power of the battery 140 into AC power.

In an embodiment, the controller 110, the sensing unit 120, the output unit 130, the user input unit 160, the memory 170, the communication unit 180, and the driving unit 190 may receive power from the battery 140 to perform functions. Although not shown in FIG. 1, the inhaler 100 may further include a power conversion circuit, for example, a low dropout (LDO) circuit or a voltage regulator circuit, which converts power of the battery 140 and supplies the power to respective components.

In an embodiment, the heater 150 may be formed of any suitable electrically resistive material. The suitable electrically resistive material may be, for example, a metal or a metal alloy including titanium, zirconium, tantalum, platinum, nickel, cobalt, chromium, hafnium, niobium, molybdenum, tungsten, tin, gallium, manganese, iron, copper, stainless steel, nichrome, or the like. However, embodiments are not limited thereto. In addition, the heater 150 may be implemented as a metal heating wire, a metal heating plate on which an electrically conductive track is arranged, a ceramic heating element, or the like. However, embodiments are not limited thereto.

In an embodiment, the heater 150 may be an induction heater. For example, the heater 150 may include a susceptor that heats the aerosol generating material by generating heat through a magnetic field applied by a coil.

In an embodiment, the heater 150 may include a plurality of heaters. For example, the heater 150 may include a first heater for heating an aerosol generating article and a second heater for heating a liquid.

In an embodiment, the user input unit 160 may receive information input from the user or may output information to the user. For example, the user input unit 160 may include a keypad, a dome switch, a touchpad (e.g., a contact capacitive type, a pressure resistive film type, an infrared sensing type, a surface ultrasonic conduction type, an integral tension measurement type, a piezo effect method, etc.), a jog wheel, a jog switch, or the like. However, embodiments are not limited thereto. In addition, although not shown in FIG. 1, the inhaler 100 may further include a connection interface, such as a universal serial bus (USB) interface, and may be connected to another external device through the connection interface such as a USB interface to transmit and receive information or to charge the battery 140.

In an embodiment, the memory 170, which is hardware for storing various pieces of data processed in the inhaler 100, may store data processed by the controller 110 and data to be processed thereby. The memory 170 may include at least one type of storage medium of a flash memory type memory, a hard disk type memory, a multimedia card micro type memory, a card type memory (e.g., an SD or xD memory), random access memory (RAM), static random access memory (SRAM), read-only memory (ROM), electrically erasable programmable read-only memory (EEPROM), programmable read-only memory (PROM), a magnetic memory, a magnetic disk, or an optical disk.

In an embodiment, the memory 170 may store an operating time of the inhaler 100, the maximum number of puffs, the current number of puffs, at least one temperature profile, data associated with a smoking pattern of a user, or the like.

In an embodiment, the communication unit 180 may include at least one component for communicating with another electronic device. For example, the communication unit 180 may include a short-range wireless communication unit 182 and a wireless communication unit 184.

In an embodiment, the short-range wireless communication unit 182 may include a Bluetooth communication unit, a Bluetooth Low Energy (BLE) communication unit, a near field communication unit, a wireless local area network (WLAN) communication unit (e.g., Wi-Fi), a

ZigBee communication unit, an infrared data association (IrDA) communication unit, a Wi-Fi Direct (WFD) communication unit, an ultra-wideband (UWB) communication unit, and an Ant+ communication unit. However, embodiments are not limited thereto.

In an embodiment, the wireless communication unit 184 may include a cellular network communicator, an Internet communicator, a computer network (e.g., a local area network (LAN) or a wide-area network (WAN)) communicator, or the like. However, embodiments are not limited thereto. The wireless communication unit 184 may use subscriber information (e.g., international mobile subscriber identity (IMSI)) to identify and authenticate the inhaler 100 in a communication network.

In an embodiment, the driving unit 190 may include various driving devices to assist an inhalation operation of the inhaler 100 of the user. For example, the driving unit 190 may include a vibrating member 191 to assist the transfer of powder of the inhaler 100.

In an embodiment, the vibrating member 191 may be implemented as an electronic vibrator, and when a voltage (e.g., an AC voltage) is applied, may generate vibration in response thereto. However, embodiments are not limited thereto, and the driving unit 190 may further include elements such as a motor, a shaft, a plurality of pinions, or a hydraulic device.

In an embodiment, the controller 110 may control the overall operation of the inhaler 100. In an embodiment, the controller 110 may include at least one processor. The processor may be implemented as an array of a plurality of logic gates, or may be implemented as a combination of a general-purpose microprocessor and a memory in which a program executable by the microprocessor is stored. In addition, it is to be understood by one of ordinary skill in the art to which the present disclosure pertains that it may be implemented in other types of hardware.

In an embodiment, the controller 110 may control the temperature of the heater 150 by controlling the supply of power from the battery 140 to the heater 150. For example, the controller 110 may control the supply of power by controlling switching of a switching element between the battery 140 and the heater 150. In another example, a direct heating circuit may control the supply of power to the heater 150 according to a control command from the controller 110.

In an embodiment, the controller 110 may analyze a sensing result obtained by the sensing of the sensing unit 120 and control processes to be performed thereafter. For example, the controller 110 may control power supplied to the heater 150 so that an operation of the heater 150 or the driving unit 190 starts or ends based on the sensing result obtained by the sensing of the sensing unit 120.

For example, the controller 110 may control the amount of power to be supplied to the heater 150 and the time for which the power is to be supplied, such that the heater 150 may be heated up to a predetermined temperature or maintained at an appropriate temperature, based on the sensing result obtained by the sensing of the sensing unit 120.

In an embodiment, the controller 110 may control the output unit 130 based on the sensing result obtained by the sensing of the sensing unit 120. For example, when the number of puffs counted through the puff sensor 126 reaches a preset number, the controller 110 may inform the user that the inhaler 100 is to end use thereof soon through at least one of the display 132, the haptic portion 134, or the sound outputter 136. Alternatively, for example, the puff sensor 126 may sense an inhalation state of the user and the controller 110 may control driving of the vibrating member 191 of the driving unit 190 based on this.

In an embodiment, the controller 110 may control the power supply time and/or the power supply amount for the heater 150 according to a state of a stick or a capsule sensed by the sensing unit 120.

An embodiment may also be implemented in the form of a recording medium including instructions executable by a computer, such as a program module executable by the computer. A computer-readable medium may be any available medium that can be accessed by a computer and includes a volatile medium, a non-volatile medium, a removable medium, and a non-removable medium. In addition, the computer-readable medium may include both a computer storage medium and a communication medium. The computer storage medium includes all of a volatile medium, a non-volatile medium, a removable medium, and a non-removable medium implemented by any method or technology for storage of information such as computer-readable instructions, data structures, program modules or other data. The communication medium typically includes a computer-readable command, a data structure, or other data regarding a modulated data signal such as a program module, or other transmission mechanisms, and includes an arbitrary information transfer medium.

FIG. 2 is a diagram schematically illustrating an inhaler 200 according to an embodiment.

Referring to FIG. 2, the inhaler 200 according to an embodiment may include at least one of a holder 210 and the stick 230.

In an embodiment, the holder 210 may be configured in a cylindrical or polygonal column shape. An insertion groove 215 for inserting the stick 230 may be formed in the holder 210, and the stick 230 may be inserted into the insertion groove 215 in a first direction (e.g., the −Y direction).

In an embodiment, the holder 210 may include a first surface 211, a second surface 212, and a side surface 213. The insertion groove 215 may be formed in the first surface 211 and the second surface 212 may be a surface opposite to the first surface 211. The side surface 213 may be formed between the first surface 211 and the second surface 212.

In an embodiment, the insertion groove 215 may be a recess formed on the first surface 211.

For example, the insertion groove 215 may have a shape extending along the longitudinal axis (e.g., the +/−Y direction) of the holder 210. The stick 230 may be inserted into the holder 210 in a direction (e.g., the −Y direction or the ‘first direction’) in which the insertion groove 215 extends from the first surface 211.

In an embodiment, an inlet (not shown) through which air from the outside of the holder 210 may flow into the insertion groove 215 may be formed between the outside of the holder 210 and the insertion groove 215.

Although not shown in the drawing, the holder 210 may accommodate various components of the inhaler 200 therein, for example, the holder 210 may accommodate at least a portion of a controller (e.g., the controller 110 of FIG. 1), at least one sensor (e.g., the sensing unit 120 of FIG. 1), and a battery (e.g., the battery 140 of FIG. 1).

In an embodiment, the stick 230 may be configured in a cylindrical or polygonal column shape and may have a size and a shape that may be inserted into the insertion groove 215 of the holder 210. The stick 230 may accommodate powder P therein.

In an embodiment, a mouthpiece 231 may be provided on one end of the stick 230. For example, the mouthpiece 231 may be positioned opposite to an area (e.g., a chamber 233 of FIG. 3a) where the stick 230 is inserted into the insertion groove 215. The user may inhale air by applying negative pressure to the stick 230. For example, the user may inhale the powder P, or air or aerosol containing the powder P while holding the mouthpiece 231 in the mouth of the user.

FIG. 3a is a diagram schematically illustrating the inside of the inhaler 200 according to an embodiment and FIG. 3b is a diagram schematically illustrating the inside of the inhaler 200 according to an embodiment.

Specifically, FIGS. 3a and 3b are diagrams illustrating the inside of area A shown in FIG. 2. FIG. 3a illustrates a state in which the stick 230 is partially inserted into or being inserted into the insertion groove 215 of the holder 210, and FIG. 3b illustrates a state in which the stick 230 is substantially completely inserted into the insertion groove 215 of the holder 210.

Referring to FIGS. 3a and 3b, the inhaler 200 according to an embodiment may include at least one of a piercing member 220, an elastic member 225, the chamber 233, and a piercing hole 234.

In an embodiment, the stick 230 may include the chamber 233 to accommodate the capsule 232. The chamber 233 may be a partial area of the stick 230 that is inserted into the insertion groove 215. The chamber 233 may be a space for accommodating or storing the capsule 232 or may be a space for limiting the movement of the capsule 232.

In an embodiment, the capsule 232 may contain the powder P therein. The powder P may be a tobacco extract in a small particle state, or the powder P may be a pharmacological material such as caffeine, taurine, aspirin, sedatives, sleeping pills, bronchodilators, or vaccines, or a composition or a functional material containing a material such as free-nicotine, or nicotine salt. However, this is only an example, and the powder P in the capsule 232 may be replaced with liquid, gas, or a combination of some of these.

In an embodiment, the piercing hole 234 may be an opening that opens toward the chamber 233, and thus exposes the chamber 233 to the outside of the stick 230. The piercing hole 234 may be formed in a surface of the stick 230 facing the insertion groove 215, desirably in an area facing the piercing member 220. The piercing hole 234 may have a diameter that is greater than or equal to that of the piercing member 220.

In an embodiment, the stick 230 may include an airflow channel 235 that provides fluid communication between the chamber 233 and a mouthpiece (e.g., the mouthpiece 231 of FIG.

2). The airflow channel 235 may be a flow path through which air containing the powder P flows, and the airflow channel 235 and the chamber 233 may be partitioned by a mesh 236.

In an embodiment, the mesh 236 may pass the powder P and air and may limit the passage of the capsule 232 or other foreign materials. Also, the mesh 236 may filter some of the powder P or help the powder P not to agglomerate. For example, the diameter of a single hole of the mesh 236 may be 5 micrometers.

In an embodiment, when the capsule 232 is crushed, at least a portion of the powder P in the capsule 232 may be discharged to the chamber 233. When the user inhales air of the stick 230 through the mouthpiece 231, the powder P may pass through the mesh 236, move to the mouthpiece 231 through the airflow channel 235, and be inhaled by the user.

In an embodiment, the stick 230 may be disposable and may be replaced when the powder P is exhausted. Alternatively, the stick 230 may be reusable, and when the powder P is exhausted, the stick 230 may be refilled with the capsule 232 or the powder P and used again.

In an embodiment, the piercing member 220 may be provided in the insertion groove 215 and may protrude from the insertion groove 215 in a direction (e.g., the +Y direction) facing the stick 230. The piercing member 220 may crush the capsule 232. For example, when the stick 230 is inserted into the insertion groove 215 in the first direction (e.g., the −Y direction), at least a partial area of the piercing member 220 may penetrate the piercing hole 234 and be inserted into the chamber 233 of the stick 230, thereby partially crushing the capsule 232.

In an embodiment, the end portion of the piercing member 220 may have a sharp or pointed shape. For example, the piercing member 220 may be a needle or a sting. The end portion of the piercing member 220 may crush a partial area of the capsule 232 and form the perforation in the capsule 232. The capsule 232 may discharge the powder P into the chamber 233 through the perforation crushed by the piercing member 220.

The elastic member 225 according to an embodiment may be provided in the insertion groove 215. When the stick 230 is inserted into the insertion groove 215, the elastic member 225 may be deformed (e.g., compressed) by the stick 230. When the elastic member 225 is deformed, the elastic member 225 may press the stick 230 in a direction (e.g., the +Y direction) opposite to the first direction by the elastic force. The elastic member 225 may include a coil spring that may apply the elastic force.

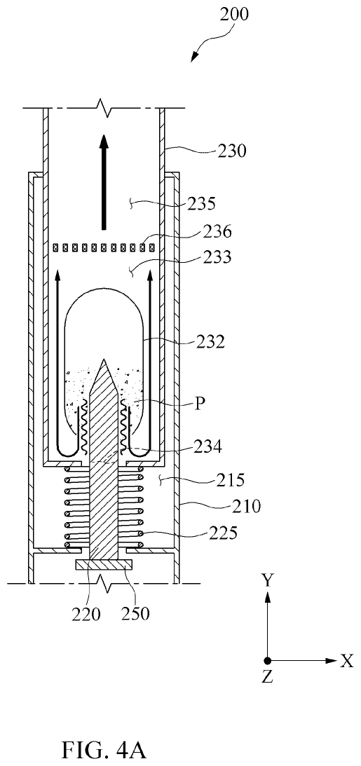

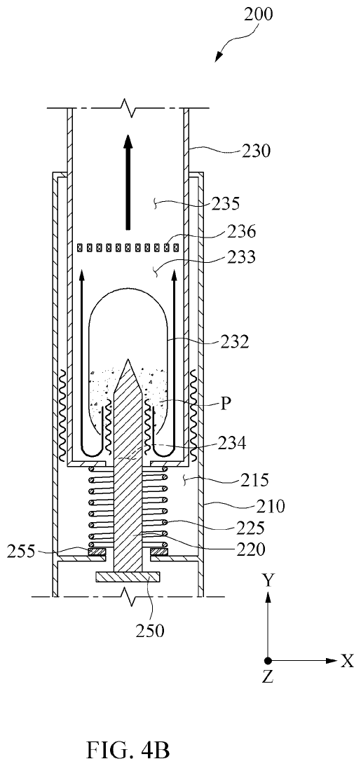

FIG. 4a is a diagram schematically illustrating the inside of the inhaler 200 according to an embodiment.

Referring to FIG. 4a, the inhaler 200 according to an embodiment may include a vibrating member 250 (e.g., the vibrating member 191 of FIG. 1).

In an embodiment, when the capsule 232 is crushed by the piercing member 220, the perforation communicating with the chamber 233 is formed in the capsule 232 and at least a portion of the powder P in the capsule 232 may be discharged into the chamber 233 through the perforation. The powder P discharged into the chamber 233 may pass through the mesh 236, be transmitted to the airflow channel 235, pass through the airflow channel 235, pass through a mouthpiece (e.g., the mouthpiece 231 of FIG. 2), and be inhaled by the user.

In an embodiment, based on various parameters (e.g., the amount of the powder P discharged per unit time, the density of the powder P in the air passing through the airflow channel 235, or the degree of spread of the discharged powder P) associated with the powder P discharged from the capsule 232, the amount of the powder P that the user inhales may also vary.

In an embodiment, by controlling the amount of the powder P (hereinafter, referred to as the ‘amount of discharge of the powder P’) discharged from the capsule 232 per unit time through the vibrating member 250, the inhaler 200 may provide the powder P to the user in accordance with the condition of the user, the use environment, or the preference of the user.

In an embodiment, the vibrating member 250 may provide vibration to the piercing member 220. Alternatively, the vibrating member 250 may directly or indirectly vibrate at least one of the capsule 232, the powder P, or the chamber 233. Hereinafter, for convenience of description, an embodiment of the inhaler 200 is described based on the vibrating member 250 that vibrates the piercing member 220 with reference to the drawings.

In an embodiment, the vibrating member 250 may be implemented as an electronic vibrator that generates vibration when a voltage (e.g., an AC voltage) is applied to electronic vibrator. However, embodiments are not limited thereto and may be implemented in various structures and configurations capable of providing vibration to the piercing member 220.

In an embodiment, the vibrating member 250 may assist the discharge of the powder P of the capsule 232 by applying vibration to the piercing member 220. When the vibrating member 250 vibrates the piercing member 220, the perforation formed in the capsule 232 becomes bigger, or the vibration may be transmitted to the capsule 232 or the powder P. As a result, the amount of discharge of the powder P may increase.

The vibrating member 250 according to an embodiment may vibrate the piercing member 220 in a direction in which the stick 230 is inserted into the holder 210, that is, a direction (e.g., the +/−Y direction) substantially parallel to the first direction (e.g., the −Y direction).

In an embodiment, when the vibrating member 250 vibrates in a direction substantially parallel to the first direction, the powder P positioned between the capsule 232 and the piercing member 220 and the stagnant powder P may be effectively discharged. Further, there is an advantage that the diameter of the piercing hole 234 does not have to be increased to secure the vibration radius of the piercing member 220. In this regard, the powder P may be prevented from being discharged to the outside of the stick 230 through the piercing hole 234. In addition, the vibration of the vibrating member 250 may be transmitted mainly to the piercing member 220 and the capsule 232, and the vibration to the stick 230 or the holder 210 may be reduced or prevented.

Alternatively, the vibrating member 250 according to an embodiment may vibrate the piercing member 220 in a direction in which the stick 230 is inserted into the holder 210, that is, a direction (e.g., the X-Z plane direction) substantially perpendicular to the first direction (e.g., the −Y direction). The vibration of the piercing member 220 may be transmitted to the capsule 232, and as the capsule 232 vibrates, the discharge of the powder P may be promoted.

In an embodiment, when the vibrating member 250 vibrates in a direction substantially perpendicular to the first direction, the piercing member 220 may increase the size of the perforation, and thus the piercing member 220 may more effectively discharge the powder P by securing a space between the capsule 232 and the piercing member 220. Further, the capsule 232 may repeatedly collide with the chamber 233 by vibration, and through this, the amount of discharge of the powder P may further increase.

The vibrating member 250 according to an embodiment may vibrate the piercing member 220 in a direction substantially parallel to the first direction and in a direction substantially perpendicular to the first direction. As the vibrating member 250 vibrates the capsule 232 three-dimensionally through the piercing member 220, the amount of discharge of the powder P may be relatively increased compared to the simple reciprocating motion.

In various embodiments, the inhaler 200 may need to reduce or increase the amount or rate of discharge of the powder P discharged from the capsule 232 according to various factors such as the respiration volume of the user or the preference of the user. In the case of a single size of the perforation, it may be difficult to meet the various environments and various needs of the user.

For example, when the perforation is large, a lot of the powder P is discharged in a short period of time, so the density of the powder P that the user inhales may be irregular or greatly increase. Alternatively, when the perforation is small, it is difficult to discharge a sufficient amount of the powder P, so a user with low lung capacity may have difficulty inhaling the powder P. According to an embodiment, the vibrating member 250 may control the powder P to be efficiently discharged from the capsule 232 by providing vibration to the capsule 232.

The inhaler 200 according to various embodiments of the present disclosure may control the inhalation density of the powder P of the user by forming a relatively small-sized perforation in the capsule 232 through the piercing member 220 and controlling the amount of discharge of the powder P discharged from the capsule 232 per unit time through the vibrating member 250.

FIG. 4b is a diagram schematically illustrating the inside of the inhaler 200 according to an embodiment.

Referring to FIG. 4b, the inhaler 200 according to an embodiment may include a sub-vibrating member 255 (e.g., the vibrating member 191 of FIG. 1).

In the description of FIG. 4b, a duplicate description with the above description for the inhaler 200 is omitted.

In an embodiment, the sub-vibrating member 255 may provide vibration to the chamber 233. The sub-vibrating member 255 may directly or indirectly vibrate the chamber 233, thereby assisting the powder P discharged into the chamber 233 in moving to the airflow channel 235. The sub-vibrating member 255 may assist the vibrating member 250 in promoting the discharge of the powder P from the capsule 232.

In an embodiment, as shown in FIG. 4b, the sub-vibrating member 255 may be connected to the elastic member 225 and provide vibration to the chamber 233 through the elastic member 225. However, embodiments are not limited thereto, and the sub-vibrating member 255 may be implemented in various structures for transmitting vibration to the chamber 233. For example, the sub-vibrating member 255 may be directly connected to the stick 230 or arranged on the holder 210 such that the sub-vibrating member 255 can impact the stick 230.

In an embodiment, the chamber 233 may provide kinetic energy to the powder P in the chamber 233 by vibration. As such, the powder P may be evenly spread by the vibration of the chamber 233 and move along the airflow. Also, the vibration of the chamber 233 may be transmitted to the capsule 232 and the sub-vibrating member 255 may promote the discharge of the powder P by vibrating the capsule 232.

The sub-vibrating member 255 according to an embodiment may vibrate the chamber 233 in a direction in which the stick 230 is inserted into the holder 210, that is, a direction (e.g., the +/−Y direction) substantially parallel to the first direction (e.g., the −Y direction). In this case, the elastic member 255 may assist and strengthen the vibration of the sub-vibrating member 255. As the chamber 233 vibrates in a direction substantially horizontal to the first direction, the powder P remaining on the bottom surface of the chamber 233 may be effectively moved. In this regard, there is no need to separately increase an size of the insertion groove 215 for the vibration of the chamber 233.

The sub-vibrating member 255 according to an embodiment may vibrate the chamber 233 in a direction in which the stick 230 is inserted into the holder 210, that is, a direction (e.g., the X-Z plane direction) substantially perpendicular to the first direction. In this case, the chamber 233 may hit the capsule 232 repeatedly and thus effectively induces the discharge of the powder P. Also, as the chamber 233 vibrates in a direction substantially perpendicular to the first direction, the powder P may be evenly spread in a substantially horizontal direction of the chamber 233.

The sub-vibrating member 255 according to an embodiment may vibrate the chamber 233 in a direction substantially parallel to the first direction and in a direction substantially perpendicular to the first direction. The sub-vibrating member 255 may relatively further increase the amount of discharge of the powder P by vibrating the chamber 233 three-dimensionally, compared to the simple reciprocating motion.

In an embodiment, a processor (e.g., the processor 110 of FIG. 1) may acquire information about driving of the inhaler 200 from various types of sensors (e.g., the sensing unit 120 of FIG. 1), and based on this, may change various parameters such as the number of vibrations, the vibration intensity, and the vibration time of the vibrating member 250 and/or the sub-vibrating member 255, thereby controlling the vibration.

For example, the processor 110 may receive information about the airflow inside the stick 230 through a puff sensor (e.g., the puff sensor 126 of FIG. 1), and when it is determined that the airflow is insufficient, the processor 110 may increase the amount of discharge of the powder P by increasing the vibration intensity or the number of vibrations of the vibrating member 250 and/or the sub-vibrating member 255.

Alternatively, the processor 110 may receive a separate input signal from a user input unit (e.g., the user input unit 160 of FIG. 1) or a communication unit (e.g., the communication unit 180 of FIG. 1), and based on this, may control the vibration of the vibrating member 250 and/or the sub-vibrating member 255.

For example, the processor 110 may control the vibration of the vibrating member 250 and/or the sub-vibrating member 255 according to various factors such as the lung capacity of the user, the preference of the user, and the use environment. The inhaler 200 according to various embodiments of the present disclosure may help the user to smoothly inhale the powder P and may provide the inhaler 200 customized for the user.

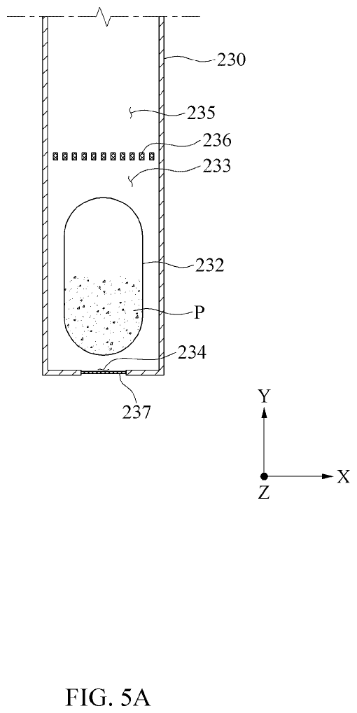

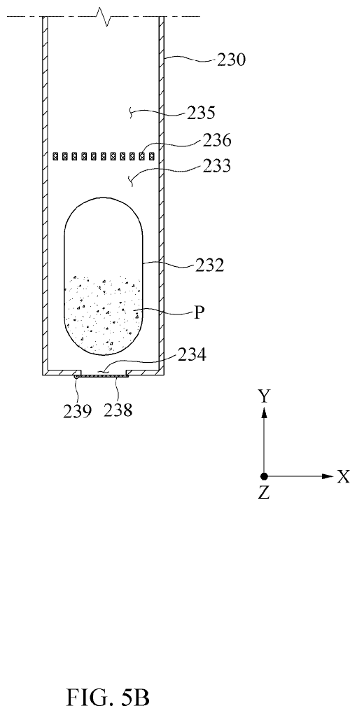

FIG. 5a is a cross-sectional view of a stick 230 according to an embodiment and FIG. 5b is a cross-sectional view of the stick 230 according to another embodiment. Specifically, FIGS. 5a and 5b are cross-sectional views of a partial area of the stick 230 in a state separated from the holder 210.

Referring to FIGS. 5a and 5b, the stick 230 may include at least one of a sealing member 237 or a door 238.

As shown in FIG. 5a, the sealing member 237 may seal the piercing hole 234. The sealing member 237 may be crushed by the piercing member 220 while the stick 230 is being inserted into the insertion groove 215. For example, the stick 230 may be disposable. Alternatively, the stick 230 may be reused by replacing the sealing member 237 and the capsule 232 after the sealing member 237 and the capsule 232 are crushed.

In an embodiment, the sealing member 237 may protect the chamber 233 from water or foreign materials flowing into the chamber 233 during the manufacturing and transportation of the stick 230. Also, the sealing member 237 may limit an area of the piercing hole 234 crushed by the piercing member 220 to prevent the powder P from being discharged to the outside of the stick 230 through the piercing hole 234.

As shown in FIG. 5b, the door 238 may selectively open and close the piercing hole 234. The door 238 may be opened before or while the stick 230 is inserted into the insertion groove 215. The door 238 may be moved by a door hinge 239.

For example, the door hinge 239 may move the door 238 in a substantially horizontal direction (e.g., the X-Z plane direction), or may tilt the door 238 in a substantially vertical direction (e.g., the +/−Y direction).

In an embodiment, the door 238 may be opened when the inhaler 200 is in use or ready for use and may be closed when the inhaler 200 is not in use. The door 238 may protect the chamber 233 from water or foreign materials flowing into the chamber 233 during the manufacturing and transportation of the stick 230. Alternatively, a used or crushed capsule 232 of the chamber 233 may be replaced through the door 238.

In an embodiment, a processor (e.g., the processor 110 of FIG. 1) may acquire information about the insertion of the stick 230 from various types of sensors (e.g., the sensing unit 120 of FIG. 1), and based on this, may control driving of the door hinge 239.

For example, the processor 110 may detect whether the stick 230 is inserted into or being inserted into the insertion groove 215 through an insertion detection sensor (e.g., the insertion detection sensor 124 of FIG. 1), and based on this, may open the door 238 by controlling the door hinge 239 so that the piercing member 220 passes through the piercing hole 234. However, embodiments are not limited thereto, and the door 238 may be manually opened and closed by the user. For example, the user may directly open and close the door 238. As another example, the inhaler 200 may have a separate switch (not shown) connected to the door 238, and the user may open and close the door 238 by controlling the switch (not shown).

While the embodiments are described with reference to drawings, it will be apparent to one of ordinary skill in the art that various alterations and modifications in form and details may be made in these embodiments without departing from the spirit and scope of the claims and their equivalents. For example, suitable results may be achieved if the described techniques are performed in a different order and/or if components in a described system, architecture, device, or circuit are combined in a different manner and/or replaced or supplemented by other components or their equivalents. Therefore, other implementations, other embodiments, and/or equivalents of the claims are within the scope of the following claims.

1. An inhaler comprising:
a stick comprising a chamber configured to accommodate a capsule containing powder and a piercing hole that opens toward the chamber;
a holder comprising an insertion groove into which the stick is inserted in a first direction;
a piercing member provided in the insertion groove and configured to crush the capsule through the piercing hole when the stick is inserted into the insertion groove; and
a vibrating member configured to provide vibration to the piercing member.

2. The inhaler of claim 1, wherein the vibrating member is configured to vibrate the piercing member in a direction substantially parallel to the first direction.

3. The inhaler of claim 1, wherein the vibrating member is configured to vibrate the piercing member in a direction substantially perpendicular to the first direction.

4. The inhaler of claim 1, wherein the vibrating member is configured to vibrate the piercing member in a direction substantially parallel to the first direction and in a direction substantially perpendicular to the first direction.

5. The inhaler of claim 1, further comprising:
a puff sensor configured to sense airflow inside the stick; and
a processor configured to receive a sensing result from the puff sensor and control vibration of the vibrating member based on the sensing result.

6. The inhaler of claim 1, further comprising:
a sub-vibrating member configured to provide vibration to the chamber.

7. The inhaler of claim 6, further comprising:
an elastic member provided in the insertion groove and configured to be pressed by the stick when the stick is inserted into the insertion groove,
wherein the sub-vibrating member is configured to provide vibration to the chamber of the stick through the elastic member.

8. The inhaler of claim 6, wherein the sub-vibrating member is configured to vibrate the chamber in a direction substantially parallel to the first direction.

9. The inhaler of claim 6, wherein the sub-vibrating member is configured to vibrate the chamber in a direction substantially perpendicular to the first direction.

10. The inhaler of claim 6, wherein the sub-vibrating member is configured to vibrate the chamber in a direction substantially parallel to the first direction and in a direction substantially perpendicular to the first direction.

11. The inhaler of claim 6, further comprising:
a puff sensor configured to sense airflow inside the stick; and
a processor configured to receive a sensing result from the puff sensor and control vibration of the vibrating member and the sub-vibrating member based on the sensing result.

12. The inhaler of claim 1, wherein the stick comprises:
a mouthpiece provided on an end portion opposite to the chamber;
an airflow channel configured to provide fluid communication between the chamber and the mouthpiece; and
a mesh arranged between the airflow channel and the chamber.

13. The inhaler of claim 1, wherein the stick further comprises a sealing member configured to seal the piercing hole and be crushed by the piercing member when the stick is inserted into the insertion groove.

14. The inhaler of claim 1, wherein the stick comprises a door configured to selectively open and close the piercing hole.

15. The inhaler of claim 14, further comprising:
an insertion detection sensor configured to detect whether the stick is inserted into the insertion groove;
a door hinge configured to move the door; and
a processor configured to receive a detection result from the insertion detection sensor and open and close the door by controlling the door hinge based on the detection result.
